(19) United States
(12) Patent Application Publication
(10) Pub. No.: US 2008/0015660 A1
(43) Pub. Date: Jan. 17, 2008

(54) Method And Apparatus For Photo-Chemical Oculoplasty/Keratoplasty

(75) Inventor: Satish V. Herekar, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: PriaVision, Inc., Menlo Park, CA (US)

(21) Appl. No.: 11/776,470

(22) Filed: Jul. 11, 2007

Related U.S. Application Data
(60) Provisional application No. 60/830,885, filed on Jul. 13, 2006.

(51) Int. Cl. A61B 18/18

(52) U.S. Cl. 607/88

(57) ABSTRACT

Method and apparatus for performing oculoplasty including applying a photosensitizer solution to a human eye surface includes defining a treatment region within the human eye surface. The treatment region is associated with a predetermined spatial pattern. The method further includes irradiating the treatment region with controlled photoactivating radiation. In relation to presbyopia treatments, shrinkage of the paralimbal scleral region near the scleral spur results in improved near focus with no loss in far acuities.

### CROSS-REFERENCES TO RELATED APPLICATIONS

See Application Data Sheet.

### STATEMENT AS TO RIGHTS TO INVENTIONS MADE UNDER FEDERALLY SPONSORED RESEARCH OR DEVELOPMENT

Not Applicable

### REFERENCE TO A “SEQUENCE LISTING,” A TABLE, OR A COMPUTER PROGRAM LISTING APPENDIX SUBMITTED ON A COMPACT DISK.

Not Applicable

### BACKGROUND OF THE INVENTION

This invention relates to ophthalmic surgery Current ophthalmic surgery frequently involves corrections utilizing cuts, ablations, implants, emulsification/aspiration and thermal coagulation of ocular tissues. Over time, all these procedures frequently result in regional structural tissue weakening. Invasive procedures such as LASIK, PRK, INTACs, CK/LTK, and RLE/IOLs, lamellar grafts, and transplants and laser/non-laser flap makers sometimes result in biomechanical weakening, intense wound healing, regression, and scar and opacifying tissue formation. These surgeries affect ocular tissues such as the cornea/lens, thereby reducing or eliminating myopia, hyperopia, presbyopia, cataracts and/or astigmatism. Current clinical outcomes result in undesirable side effects notably predictability, contrast loss, night vision, glare, halo, haze, and the like as well as lack of long term stability and in some extreme cases: keratectasia or localized corneal thinning and bulging. Touchups, to correct for inaccurate surgical outcomes, suffer from similar shortcomings. Particulate infiltrates during flap making are known to cause complications (e.g., DLK-“Sands of Sahara” and microbial infections). Dry eye complaints immediate post-op are common due to corneal nerve damage. Induced high order aberrations (HOAs), infectious keratitis, epithelial ingrowth/stromal melts, irregular astigmatism, stria/micro-wrinkles and shifted/button hole flaps have been reported in published literature. Weakening due to RK often results in induced hyperopia.

Excimer/Femtosecond techniques generally require high cost/high maintenance/large footprint systems. LTK/CK techniques are known to suffer from regression of effect due to thermal collagen denaturation.

Recent rapid prototyping/stereolithography tools have seen use of curable liquid resin surfaces illuminated by UVA DLP DMD technologies but are meant as sequential crosslinking to create custom objects, and neither non-opacification nor shrinkage of living tissue are considered as the primary desired effects, primarily rapid rigidity.

Therefore, there is a need in the art for improved methods and apparatus for modifying the refractive properties of ocular tissue.

### SUMMARY OF THE INVENTION

According to the invention, shaping or treating reactive target regions and more specifically performing structural modification of ocular tissues utilizes ultravioletiblue radiation to produce non-opacifying shrinkage and stiffening of ocular tissue. A customized, pixel-based treatment region and low to moderate ultraviolet/blue radiation fluences produces refractive modifications of eye tissue. In relation to presbyopia treatments, shrinkage of the paralimbal scleral region near the scleral spur results in improved near focus with no loss in far acuities.

According to an embodiment of the invention, a method of performing oculoplasty includes applying a photosensitizer solution to a human eye surface and defining a treatment region within the human eye surface. The treatment region is associated with a predetennined spatial pattern. The method further includes irradiating the treatment region with controlled photoactivating radiation.

According to another embodiment of the present invention, a method for treating a living tissue includes applying a photosensitizer solution to a surface of the tissue and defining a treatment region within the surface. The treatment region is associated with a predetermined spatial pattern of intensities. The method also includes irradiating the treatment region with an effective dose of controlled ultraviolet/blue radiation according to the spatial pattern.

According to yet another embodiment of the present invention, an apparatus for performing oculoplasty includes an applicator for applying a photosensitizer solution to a human eye surface. The apparatus also includes an illuminator for irradiating a defined treatment region within the human eye surface with an effective amount controlled ultraviolet/blue radiation according to a predetermined spatial pattern of intensities.

Many benefits are achieved by way of the present invention over conventional techniques. For example, embodiments of the present invention provide non-opacifying, non-invasive oculoplasty treatments. Moreover, benefits include treatment protocols using low power ultraviolet/blue light sources adapted to provide for shrinkage and stiffening of ocular tissue. Embodiments of the present invention provide a treatment utilizing a digital pixel-based treatment region that is customized for the needs of a particular patient. Depending upon the embodiment, one or more of these benefits, as well as other benefits, may be achieved. These and other benefits will be described in more detail throughout the present specification and more particularly below in conjunction with the following drawings.

### BRIEF DESCRIPTION OF THE DRAWINGS

FIG. 1A is a simplified schematic diagram of an ocular treatment system according to an embodiment of the present invention;

FIG. 1B is a simplified schematic diagram of an alternative ocular treatment system according to an alternative embodiment of the present invention

FIG. 2A is a simplified plot of absorption coefficient as a function of wavelength for different materials;

FIG. 2B is a simplified plot of relative penetration as a function of wavelength for a material;

FIG. 3 is a simplified flowchart illustrating a treatment process according to an embodiment of the present invention;

FIG. 4 is a simplified plot of absorbance as a function of wavelength for riboflavin and recombinant riboflavin;

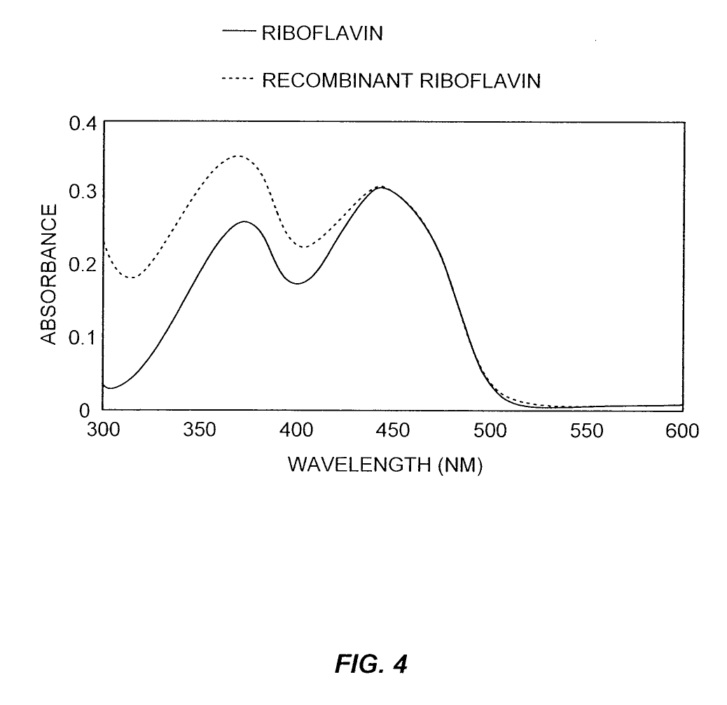

FIG. 5 is a simplified schematic diagram of another alternative ocular treatment system according to another alternative embodiment of the present invention;

### DETAILED DESCRIPTION OF THE INVENTION

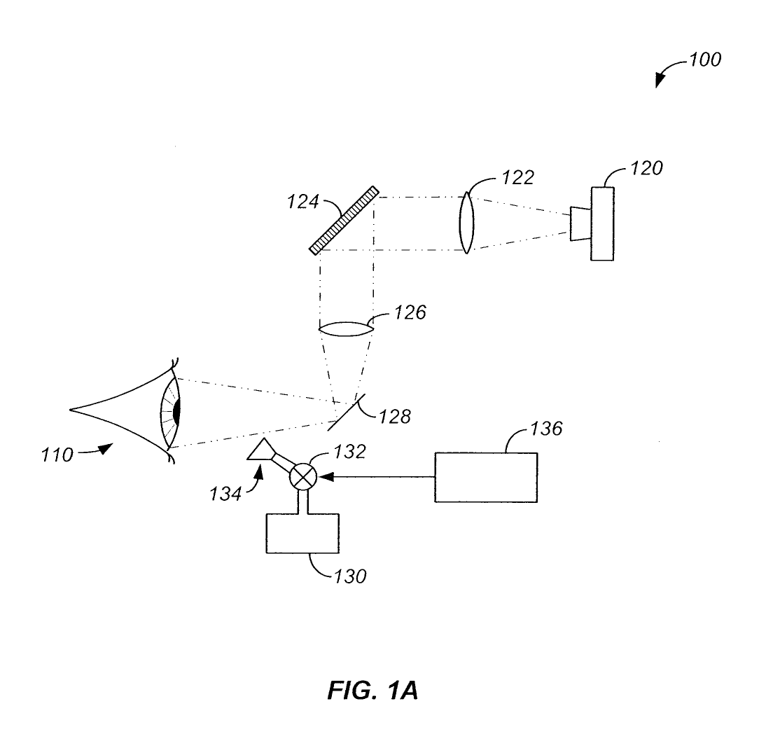

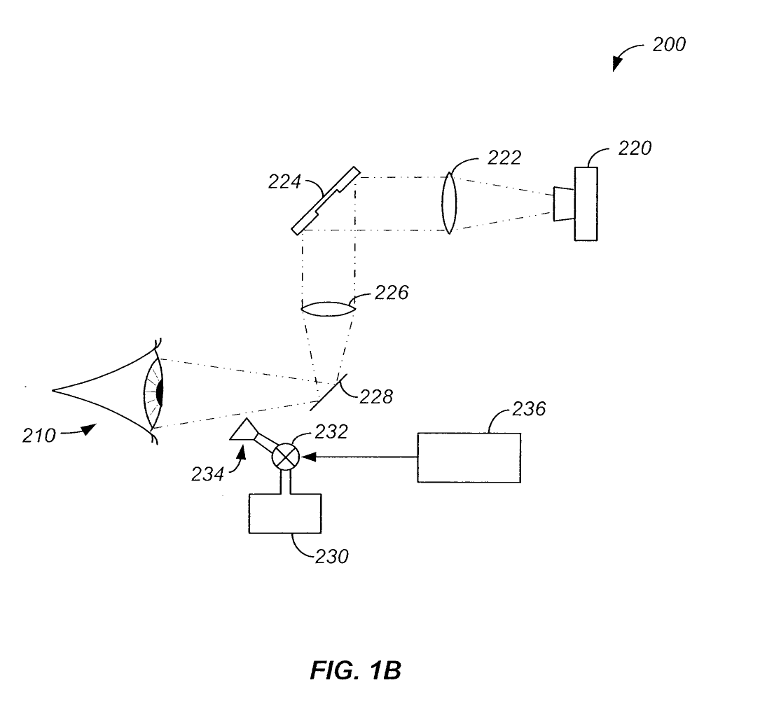

FIG. 1A is a simplified schematic diagram of an ocular treatment system according to an embodiment of the present invention. As described throughout the present specification, embodiments of the present invention utilize one or more of the following components: a spatial light modulator (SLM), for example, a DLP® system from Texas Instruments of Dallas, Tex., a PC interface, light source for treatment (e.g., a mercury arc or similar source with power stabilization control), a light source for pachymetry/wavefront sensing and the like, collimating optics, one or more filters for UVA/Visible or other wavelengths, one or more beam splitters (e.g., for a spectrophotometer, a visible/IR camera, or other monitoring apparatus), a shutter beam block, spray nozzles with multiple reservoirs mixers and temperature control, and CCD cameras/monitoring devices. The components listed above are provided merely by way of example. Additional components are provided, components are removed, and/or multiple instances of some components are provided in alternative embodiments. One of ordinary skill in the art would recognize many variations, modifications, and alternatives.

As illustrated in FIG. 1A, the ocular treatment system 100 includes apparatus adapted to provide treatments for human eye 110. This diagram is merely an example, which should not unduly limit the scope of the claims herein. One of ordinary skill in the art would recognize many other variations, modifications, and alternatives. Other embodiments may utilize additional or fewer components depending on the particular application.

Photosensitizer from storage tank 130 is provided through valve 132 and orifice 134 under control of electronic control system 136. Metering, dosage, timing, and other control of the photosensitizer through orifice 136 is described in additional detail in U.S. patent application Ser. No. 10/958,711, filed Oct. 4, 2004, commonly assigned and incorporated herein by reference in its entirety for all purposes. In a specific embodiment, the photosensitizer includes riboflavin.

The ocular treatment system 100 also includes an ultraviolet/blue source 120, a collimating lens 122, a pixel-based spatial light modulator 124, a projection lens 126, and a turning mirror 128. Depending on the particular embodiment, either pulsed light, CW light, or a combination thereof is utilized during treatment. Radiation from the ultraviolet/blue source is focused by collimating lens 124 to illuminate pixel-based spatial light modulator 124. Additional details of the pixel-based spatial light modulator 124 are provided throughout the present specification and more particularly below. Utilizing the ocular treatment system 100, the human eye 110 is treated with photosensitizer from storage tank 130 utilizing orifice 134 and then irradiated with a predetermined spatial pattern through control of the pixel-based spatial light modulator 124 through the use of control electronics (not shown).

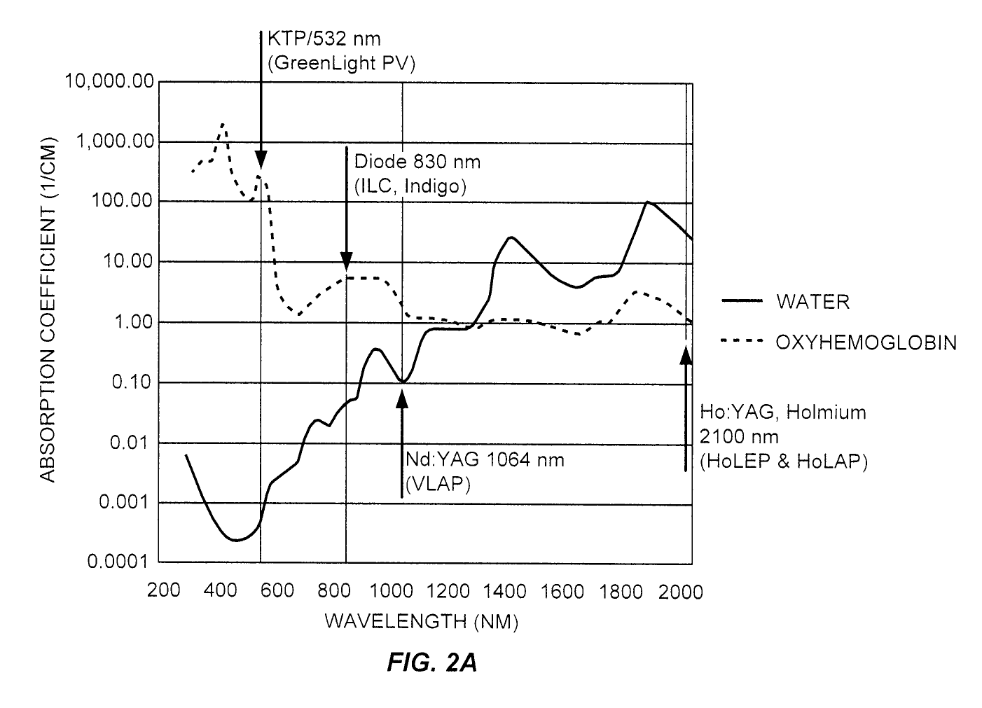

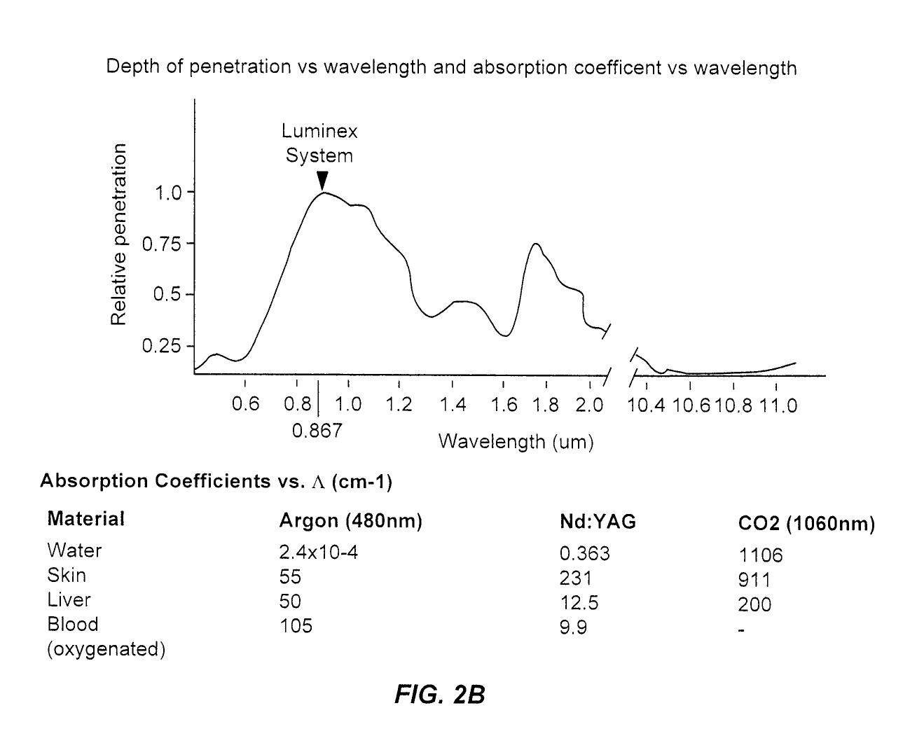

Referring to FIGS. 2A-2B, it will be appreciated that the use of a broadband source including many spectral components enables embodiments of the present invention to provide systems in which the treatment wavelength is tailored to a particular application. For example, spectral filtering of the source enables the system to operate with a predetermined absorption and penetration depth. As discussed below, enhancing fluids are utilized to temporally modify the penetration depth in a non-opacifying manner, increasing the penetration depth during treatment and returning the penetration depth to normal levels post-treatment. Thus, embodiments of the present invention provide for surface treatments as well as deeper curing treatments that are dependent, for example, on treatment wavelength.

In an embodiment, the pixel-based spatial light modulator 124 is a two-dimensional array of controllable micro-mirrors. In a specific embodiment, the controllable pixel-based array 124 has a resolution of 1,024×768 pixels with a capability of 1,000 levels of programmable “gray scale” intensity modulation. The optical system is structured to provide a pixel size of 40 μm at the focal plane aligned with a surface of the eye undergoing treatment. Embodiments of the present invention are not limited to a resolution of 1,024×768 pixels, but may utilize different pixel counts, pixel size, array geometries, and number of “gray scale” intensity levels. In alternative embodiments, delivery of photons is provided by DLP, fibers, other contact/non-contact means, combinations of these, and the like. One of ordinary skill in the art would recognize many variations, modifications, and alternatives.

Custom patterning using a customized micro-mirror based digital light projection (e.g., a DLP® projector available from Texas Instruments of Dallas, Tex.) system provides an intensity modulated (i.e., gray scale) treatment pattern over which shrinkage is produced. As illustrated in FIG. 1A, a micro-mirror based system, is used to tailor the delivery of ultraviolet/blue radiation and specifically UVA radiation to predetermined regions of the eye. Although FIG. 1A illustrates the use of a micro-mirror based projection system, other pixel-based optical projection systems are included according to embodiments of the present invention. For example, LCD-based systems, LOCOS-based systems, and the like may be utilized. One of ordinary skill in the art would recognize many variations, modifications, and alternatives.

Embodiments of the present invention as described herein utilize the surprising discovery that human tissues, and ocular tissue in particular, can be precisely reshaped and strengthened without incisions or thermal delivery or opacification and with only a photosensitizer and photonic excitation benefiting refractive correction or biomechanical modulation. Embodiments of the present invention provide methods and techniques that include oculoplasty and keratoplasty, which is a subset of oculoplasty, and non-thermal non-invasive (no-cut) molecular resizing/collagen shrinkage, refractive index and biomechanical modulation via crosslinking of ocular tissues (such as cornea, sclera, ciliary body, lens, TM, and the like) by photochemically affecting the underlying collagen. Novel lithographic exposure techniques for precise ocular patterning, controlled depth of effect, and online metered photosensitizer spraying are included according to some embodiments. For example, in an embodiment, methods and systems leverage commercial DLP® technology (e.g., as available from Texas Instruments) operating down to the UVA region and customized metered dose nasal spray technologies such as ones available from Valois. The use of a laser as a photon source is not essential as a mercury arc lamp (or LEDs or optical sources) can deliver the spectra power required for the transformation with/without fiber(s) coupling as well and also in part because of the output beam uniformity. The DLP(G) chip set can be utilized for eye tracking functions as well as topography projection functions during treatment exposure online.

Since, in some embodiments, the ocular surface is incisonless, one or more of intraoperative wavefront sensing, pachymetry/OCT, and topography monitoring are provided. Additionally, treatment regimes utilizing pulsing of photonic radiation (e.g., femtosecond pulses or longer or shorter pulses) in order to reduce average fluence but obtain maximum cross-shrinkage cleaving efficiency are provided in certain embodiments. Spray premixing, multi spraying, thermally or chemically modulating photochemicals before, during, and after therapy so as to better penetrate ocular tissue, better protect the treated/untreated tissue during exposure and result in better overall outcomes (e.g., apoptosis/opacification/hydration/regeneration/scaffolds) following exposure are also included within the scope of embodiments of the present invention. Use/combinations of, for example, anesthetics, hyperosmotic agents (e.g., a combination of DMSO and glycerol, and the like), viscoelastics, liquid collagen, pH modifiers, hydration/swelling modifiers, certain growth factors, wound healing enhancers, tissue inhibitors of metalloproteinase, tissue glue, crosslink breakers, loaded microspheres, and antioxidants in addition to photosensitizers are provided pre/intra/post exposure in certain embodiments. In some embodiments, a spectrophotometer is utilized to monitor the photosensitizer concentration present in situ and measure/characterize its remaining singlet-oxygen generating potential with feedback to the dispensing control system. Singlet oxygen can be produced by visible radiation as well as UVA/blue radiation. Moreover, other photosensitizers in addition to, in combination with, or in place of riboflavin are utilized in some embodiments. An OCT/pachymeter to monitor tissue thickness changes is provided in an embodiment of the optical system. System features include, but are not limited to, patient alignment by iris recognition or pupil tracking. Where multiple patient treatment visits (e.g., 3 months apart) or low fluence exposures or low dosage energy is preferred at one time, lowered intensity modulation of the pattern is easily projected utilizing embodiments of the present invention. Internal or external topography/wavefront/pachymetry map data or manual entry of basic desired refraction correction and the like may be inputs to this system in order to generate a correction nomogram/treatment plan.

In alternative embodiments, methods and systems to measure ocular tissue rigidity such as with the Reichert ORA or PriaVision SonicEye may be used in conjunction with embodiments of the present invention to refine the treatment plan. In addition to optical refractive/aberrational corrections/changes, ocular clarity, refractive index and stria smoothening improvements are provided by embodiments of the present invention. Lamellar grafts/lenticules can be custom preformed or treated in situ according to embodiments of the present invention. 3D tissue layers created from collagen baths/sprays placed on ocular tissue that are sequentially crosslinked are also included within the scope of the present invention. During operation, embodiments of the present invention utilize software that can deliver rapid video frame rate (e.g., at up to XGA resolutions or higher) images so that a “movie” can be “played” directly on the treatment region. The projection system is capable of direct focused delivery of any PC generated images to the cornea, lens and retina as well. In certain embodiments, such a system with single/multiple DLPs may be capable of projecting Snellen charts (e.g., near and far).

Other non-limiting applications for the technology described herein are contemplated for example: photodynamic therapy (PDT) treatments for CNV, Macular Edema, and age-related macular degeneration (AMD), in situ microfluidics (“channels”) for IOP control and the like, or in situ regional struts (INTACS like) creation, PriaVision presbyopia PACT procedure delivery system, delivery of other spectra from the multispectral light source such as green, red, infrared in addition to UVA and blue wavelengths upon filter selection, and light adjustable lens (LAL) adjustment by lenticular illumination, in situ crosslinking/patterning of any tissue/vasculature in vivo/ex vivo, systemic tissue pathogen reduction, and the like. Touchups for post LASIK, PRK, LTK/CK, INTACS, RK and lamellar or PKP surgeries are also provided according to embodiments of the present invention. Additionally some embodiments include methods and systems for donor tissue reshaping/stabilization for refractive neutral grafts.

According to embodiments of the present invention, methods and techniques to perform refractive surgery as well as presbyopic corrections of +/−3 diopters are provided. The present inventor has determined that in relation to presbyopia treatments, shrinkage of the paralimbal scleral region near the scleral spur results in improved near focus with no loss in far acuities. Embodiments of the present invention provide unique benefits, including the advantage of an incisionless, non-weakening process. The methods and systems described herein produce significant stabilization and strengthening of the ocular tissues.

The selection of the topical photosensitizer includes an analysis of potential endothelial, lenticular, and retinal damage. As described more fully throughout the present specification, the application of a riboflavin (vitamin B12) solution in the target region of the human eye 110 increases the absorption radiation in the UV-A portion of the spectrum. Generally, UV-A radiation is defined as radiation in the 320 nm-400 nm range. The inventor has performed studies that demonstrate that riboflavin fluoresces upon excitation at various wavelengths, including 375 nm and 436 nm.

In embodiments of the present invention, a photosensitizer solution is utilized that is delivered utilizing a disposable applicator that mitigates endothelial, lenticular, and retinal UVA damage. Accordingly, in-situ “struts” may be created (i.e., similar to INTACS but with no implants) for corneal dystropies/keratoconics utilizing embodiments of the present invention. Moreover, embodiments of the present invention provide scleral shrinkage for IOP reduction, PACT-presbyopia, and zonular shrinkage for lenticular aberrational or astigmatic corrections. Additionally, post LASIK flap stria reduction is also included in the treatments performed according to embodiments of the present invention. In a specific embodiment, a treatment is provided after cataract surgery that utilizes UVA curing adhesives that are illuminated using systems provided herein.

In an exemplary study performed utilizing an embodiment of the present invention, a porcine cornea was irradiated with a bowtie pattern at an approximate fluence of 12 mW/cm² at a wavelength of 365 nm. The bowtie pattern was exposed for 10 minutes. Prior to irradiation, either BSS drops (for a control) or PriaLight photosensitizer was applied to the porcine cornea. The BSS drops or the PriaLight photosensitize were dispensed at 5 minute intervals during treatment. Assuming approximately a quarter of the 1 cm diameter corneal surface area was exposed, a dosage of ˜2J total UVA was delivered. Other exemplary treatments included the disc, annulus, multi annuli, sequential annuli and text shape imprinting using this technique.

Utilizing the system illustrated in FIG. 1A, exposure of sample eyes treated with the photosensitizer resulted in a non-opacifying axial shrinkage/imprint of 100-150 μm that was uniform with no local optical transition zone/stress lines/hot spots. The treated region was characterized after treatment by a substantial increase in tissue stability (i.e., stiffness) and significant smoothening and stria reduction (i.e., a “Saran-wrap” like effect). The non-treated zones showed homogenous uniform shrinkage/stress lines 12 hours post-treatment. The samples were examined under a Veeco interferometer and under high magnification microscopes and demonstrated no surface opacification and sharp/clear uniform shrinkage zones with no hot spots.

The samples treated with the control fluid (i.e., BSS) showed no discemable shrinkage during examination using a microscope and no temperature rise (measured using a RayTech Minitemp IR laser thermoscanner) was noted at the exposure zones during and throughout all treatments.

Systems described herein are characterized by system cost significantly less than conventional refractive treatment system, such as LASIK. The cost of a laser-less refractive system using a commercially available micro-mirror-based projector will generally be less than LASIK systems. In a particular embodiment, a DLP® engine from Texas Instruments of Dallas, Tex. and costing less than $10,000 is used with an ultraviolet/blue bulb costing less than $1,000 and other system components.

In contrast with some LASIK systems, some embodiments do not utilize a highly sophisticated eye tracker. As described throughout the present specification, processes utilized by embodiments of the present invention are by nature relatively slow biochemical processes rather than an ablation or localized shrinkage process. Thus, some embodiments of the present invention reduce the need for expensive and complicated intraoperative wavefront plus topographical monitoring.

An alternative embodiment of the present invention incorporates one or more topographical sensors, wavefront sensors, and/or an eyetracker for online real-time corrections. A feedback loop is provided from these instruments to the controllable spatial light modulator in these alternative embodiments. In some embodiments with such a feedback loop, a method to derive modifications of the predetermined spatial pattern from wavefront aberration data or other data is provided that adjusts the predetermined spatial pattern during treatment in response to the measured wavefront aberration or other data. In other embodiments, corneal topography is utilized in place of or to complement the wavefront aberration data.

FIG. 1B is a simplified schematic diagram of an alternative ocular treatment system according to an alternative embodiment of the present invention. In certain applications, including myopia, hyperopia, astigmatism, HOA, PACT corrections, and the like, systems such as illustrated in FIG. 1A reduce the system cost significantly by providing a variety of predetermined illumination patterns using inexpensive pattern illuminators.

Photosensitizer from storage tank 230 is provided through valve 232 and orifice 234 under control of electronic control system 236. Metering, dosage, timing, and other control of the photosensitizer through orifice 236 is described in additional detail in previously referenced U.S. patent application Ser. No. 10/958,711. In a specific embodiment, the photosensitizer includes riboflavin.

The ocular treatment system 200 also includes an ultraviolet/blue source 220, a collimating lens 222, a pattern illuminator 224, a projection lens 226, and a turning mirror 228. Radiation from the ultraviolet/blue source 220 is focused by collimating lens 224 to illuminate pattern illuminator 224. Utilizing a particular pattern illuminator, predefined patterns may be formed on the surface of the human eye 110 undergoing treatment. Merely by way of example, if radial patterns are desired on the surface of the human eye 110, a radial pattern illuminator is utilized. In another application, controlled skrinkage of tissue at peripheral regions of the eye is performed using a pattern illuminator with an annular pattern. One of ordinary skill in the art would recognize many variations, modifications, and alternatives.

The ocular treatment system 200 illustrated in FIG. 1A provides for interchangeable pattern illuminators 224 depending on the particular application. Thus, the ocular treatment system 200 provides a solution that is lower in cost than the system utilizing a controllable pixel based spatial light modulator 124.

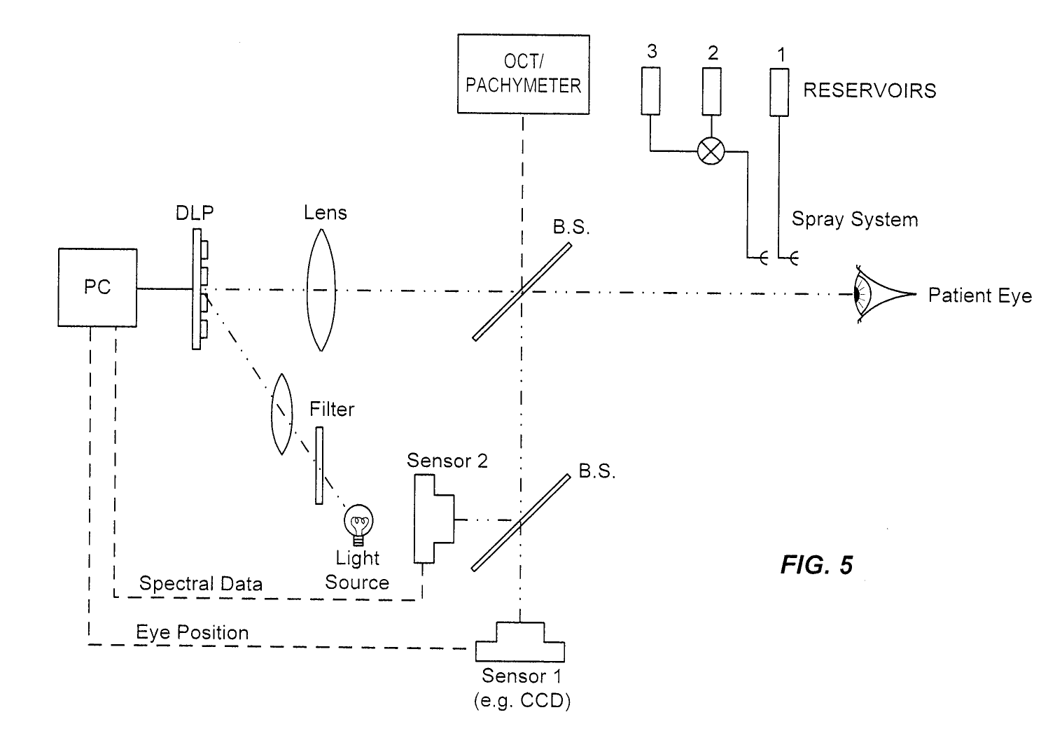

FIG. 5 is a simplified schematic diagram of another alternative ocular treatment system according to another alternative embodiment of the present invention. FIG. 5 shares some common components with the system illustrated in FIG. 1A. The system illustrated in FIG. 5 also provides additional system components including one or more sensors such as Sensor 1 and Sensor 2. In a particular embodiment, Sensor 1 is a CCD sensor that provides image data related to the eye position and Sensor 2 is a spectral sensor such as a spectrometer that provides spectral data to the PC. Also illustrates is an optical coherence tomographer (OCT)/Pachymeter coupled to the optical system. Multiple reservoirs and appropriate valving are provided for the spray system. One of ordinary skill in the art would recognize many variations, modifications, and alternatives. The system illustrated in FIG. 5 is not intended to limit the scope of embodiments of the present invention but to merely illustrate various system configurations provided within the scope of embodiments of the present invention.

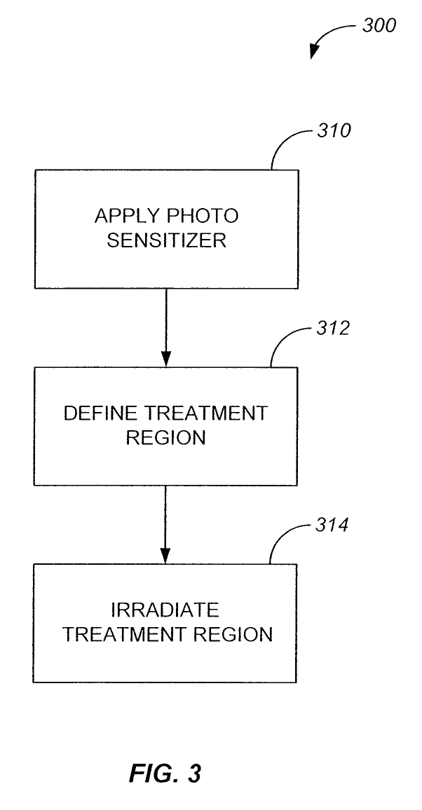

FIG. 3 is a simplified flowchart illustrating a treatment process according to an embodiment of the present invention. Treatment process 300 includes applying a photosensitizer solution to a human eye surface (310). In an embodiment, the photosensitizer solution includes riboflavin with a concentration ranging from about 0.05% to about 0.2%. The method also includes defining a treatment region within the human eye surface (312). The treatment region is associated with a predetermined spatial pattern. The method further includes irradiating the treatment region with controlled ultraviolet/blue radiation (314).

Merely by way of example, irradiation of the eye is carried out utilizing an array of micro-mirrors that provide a pixel-based output characterized by a number of selectable gray-scale intensities. In an embodiment, the number of gray-scale intensities is 1,000 or more.

It should be appreciated that the specific steps illustrated in FIG. 3 provide a particular method of performing an ocular treatment according to an embodiment of the present invention. Other sequence of steps may also be performed according to alternative embodiments. For example, alternative embodiments of the present invention may perform the steps outlined above in a different order. Moreover, the individual steps illustrated in FIG. 3 may include multiple sub-steps that may be performed in various sequences as appropriate to the individual step. Furthermore, additional steps may be added or removed depending on the particular applications. One of ordinary skill in the art would recognize many variations, modifications, and alternatives.

According to embodiments of the present invention, non-toxic antioxidants are preloaded prior to the application of the photosensitizer and are utilized to protect the endothelium/AC. These non-toxic antioxidants include: Coumarin, PENT, ALDH3A1 Vitamins C/A/E, Alpha Lipoic acid, Albumin, G6PDH Pentoic phosphate, and the like. Instrumentation to monitor the concentration of the photosensitizer at the endo/AC region is utilized in some embodiments as a real time monitor during treatment for intraoperative safety/freshness checking. As a baseline of safety threshold, Spoerl reports a 20 to 1 UVA absorption in the corneal stroma due to the riboflavin load, while Sliney has published a 1 mW/cm² continuous(16 minutes maximum time) exposure at 365 nm (without any riboflavin loading). Based on these results, we have inferred that up to a 20 mW/cm² “continuous” UVA threshold is acceptable if the cornea is fully riboflavin loaded.

Embodiments of the present invention are applicable to a wide variety of applications including in-situ INTACS creation by corneal/ocular crosslinking of tissue to improve biomechanical stability by a factor or 2-4 with no implants; presbyopic pseudophakia corrections with scleral/zonular UVA shrinkage and ciliary body translocation; PACT-UVA; lenticular aberrational corrections; IOL, ICL adjustments; glaucoma treatment for IOP reduction by shrinkage at the scleral spur trabecular meshwork; pre-post LASIK for prophylactic treatments and for reduced regression. Embodiments of the present invention provide all known benefits of KeraCure such as: Keratoconus, PMD, Corneal Dystrophy, Ulcers, and the like. Additional discussion of the KeraCure process are provided in previously referenced U.S. patent application Ser. No. 10/958,711.

The results of several studies performed by the inventors are described below. For these studies, measured fluence was 375 nm at 12 mW/cm²-15 mW/cm² and 436 nm at 130 mW/cm², soak period pre-exposure was 5 minutes and intra-exposure drops were instilled every 3 minutes, porcine eyes were not deepithelialized. Exposure durations varied from 10 minutes to 45 minutes in these exemplary studies.

Exemplary study #1 was conducted, in part, to demonstrate the feasibility of precise patterned regional corneal shrinkage with photosensitized in comparison with non-photosensitized illumination using UVA/blue wavelengths. Merely by way of example, 10 porcine eyes with control BSS loading and 10 samples with PriaLight loading were patterned with a bowtie or a circular pattern for 10-40 minutes.

Exemplary study #2 was conducted, in part, to demonstrate lithographing of complex patterns in the form of text. Text including “PRIA” and “HELLO” were lithographed by PriaLight photosensitizer+UVA/blue exposure.

Exemplary study #3 was conducted, in part, to demonstrate refractive corneal modifications with UVA/blue patterned PriaLight photosensitizer shrinkage. Merely by way of example, 12 porcine eyes were loaded with PriaLight and patterned with discs, discs with a transition zones, annulus, annuli, time sequential annuli, recorded topography, and the like.

While the present invention has been described with respect to particular embodiments and specific examples thereof, it should be understood that other embodiments may fall within the spirit and scope of the invention. The scope of the invention should, therefore, be determined with reference to the appended claims along with their full scope of equivalents.

What is claimed is:

1. A method for performing oculoplasty, the method comprising:
applying a photosensitizer solution to a human eye surface;
defining a treatment region within the human eye surface, the treatment region being associated with a predetermined spatial pattern; and
irradiating the treatment region with controlled photoactivating radiation.

2. The method of claim 1 wherein the photoactivating radiation comprises ultraviolet/blue radiation.

3. The method of claim 1 wherein the predetermined spatial pattern is defined by directed transmission of the ultraviolet/blue radiation.

4. The method of claim 3 wherein the irradiating step includes directing the ultraviolet/blue radiation of a predetermined wavelength range, with preselected intensity of a range of radiation intensities, at a plurality of locations within the spatial pattern.

5. The method of claim 4 wherein the irradiating step is carried out through a medium of an array of micro-mirrors.

6. The method of claim 5 wherein micro-mirrors of the array of micro-mirrors yield output at a plurality of selectable gray-scale intensities.

7. The method of claim 1 wherein the photosensitizer comprises a solution including riboflavin.

8. The method of claim 7 wherein concentration of riboflavin ranges from 0.05% to 0.2%.

9. The method of claim 8 wherein the concentration of riboflavin is substantially 0.1%.

10. The method of claim 1 wherein the human eye surface is a corneal region.

11. The method of claim 1 wherein the predetermined spatial pattern is a two-dimensional pattern.

12. The method of claim 11 wherein the two-dimensional pattern comprises a raster scanned pattern.

13. The method of claim 12 wherein the raster scanned pattern comprises at least 1024×868 pixels.

14. The method of claim 13 wherein a spatial resolution of each micro-mirror is substantially 20 Mm.

15. The method of claim 1 wherein the controlled ultraviolet/blue radiation has a predetermined wavelength range of 350 nm to 380 nm.

16. The method of claim 1 wherein the ultraviolet/blue radiation has an optical fluence of less than 20 mW/cm².

17. The method of claim 16 wherein the optical fluence is less than 15 mW/cm².

18. A method for treating a living tissue, the method comprising:
applying a photosensitizer solution to a surface of the tissue;
defining a treatment region within the surface, the treatment region being associated with a predetermined spatial pattern of intensities; and
irradiating the treatment region with an effective dose of controlled ultraviolet/blue radiation according to the spatial pattern.

19. An apparatus for performing oculoplasty, the apparatus comprising:
an applicator for applying a photosensitizer solution to a human eye surface; and
an illuminator for irradiating a defined treatment region within the human eye surface with an effective amount controlled ultraviolet/blue radiation according to a predetermined spatial pattern of intensities.
